(19) United States
(12) Patent Application Publication
(10) Pub. No.: US 2013/0096446 A1
(43) Pub. Date: Apr. 18, 2013

(54) Method and System for Differentiating Between Supraventricular Tachyarrhythmia and Ventricular Tachyarrhythmia

(71) Applicants: Queen's University at Kingston, Kingston (CA); Kington General Hospital, Kingston (CA)

(72) Inventors: Kevin A. Michael, Kingston (CA); Damian P. Redfearn, Kingston (CA)

(73) Assignees: KINGSTON GENERAL HOSPITAL, Kingston (CA); QUEEN'S UNIVERSITY AT KINGSTON, Kingston (CA)

(21) Appl. No.: 13/645,877

(22) Filed: Oct. 5, 2012

(30) Foreign Application Priority Data
Oct. 5, 2011 (CA) 2754429

(51) Int. Cl. A61B 5/04; A61N 1/39

(52) U.S. Cl. CPC A61B 5/04012; A61N 1/3987
USPC 600/510; 607/7

(57) ABSTRACT

A method of differentiating between supraventricular tachyarrhythmia (SVT) and ventricular tachyarrhythmia (VT) is disclosed. A post pacing interval (PPI) is determined based on a biomarker dataset. The post pacing interval is statistically analyzed relative to a threshold to differentiate between SVT and VT. A further method of differentiating between SVT and VT is disclosed. A PPI is determined based on a biomarker dataset. A tachycardia cycle length (TCL) is also determined based on the biomarker dataset. A difference of the PPI minus the TCL is statistically analyzed relative to a threshold to differentiate between SVT and VT. A non-transitory computer readable medium and a system are also disclosed for differentiating between SVT and VT.

### RELATED APPLICATION

This application claims the benefit of the filing date of U.S. Application Ser. No. 61/543,704, filed Oct. 5, 2011, the contents of which are incorporated herein by reference in their entirety.

### FIELD

The claimed invention relates to the assessment and diagnosis of the heart, and more particularly to methods and systems for differentiating between supraventricular tachyarrhythmia (SVT) and ventricular tachyarrhythmia (VT).

### BACKGROUND

The human heart 20, schematically illustrated in FIG. 1, has four contractile chambers which work together to pump blood throughout the body. The upper chambers are called atria, and the lower chambers are called ventricles. The right atrium 22 receives blood 24 that has finished a tour around the body and is depleted of oxygen. This blood 24 returns through the superior vena cava 26 and inferior vena cava 28. The right atrium 22 pumps this blood through the tricuspid valve 30 into the right ventricle 32, which pumps the oxygen-depleted blood 24 through the pulmonary valve 34 into the right and left lungs 36, 38. The lungs oxygenate the blood, and eliminate the carbon dioxide that has accumulated in the blood due to the body's many metabolic functions. The oxygenated blood 40 returns from the right and left lungs, 36, 38 and enters the heart's left atrium 42, which pumps the oxygenated blood 40 through the bicuspid valve 44 into the left ventricle 46. The left ventricle 46 then pumps the blood 40 through the aortic valve 48 into the aorta 50 and back into the blood vessels of the body. The left ventricle 46 has to exert enough pressure to keep the blood moving throughout all the blood vessels of the body. The heart is a complex and amazing organ which everyone relies on to remain healthy for a good quality of life.

During each heartbeat, the two upper chambers of the heart (atria 22, 42) contract, followed by the two lower chambers (ventricles 32, 46). The timing of the heart's contractions is directed by electrical impulses generated in the heart. When the contractions are synchronized properly, the heart pumps efficiently. The heart's electrical impulse begins in the sinoatrial (SA) node 52, located in the right atrium 22. Normally, the SA node 52 adjusts the rate of impulses, depending on the person's activity. For example, the SA node 52 increases the rate of impulses during exercise and decreases the rate of impulses during sleep. When the SA node 52 fires an impulse, electrical activity spreads through the right atrium 22 and left atrium 42, causing them to contract and force blood into the ventricles 32 and 46, respectively. The impulse travels to the atrioventricular (AV) node 54, located in the septum (near the middle of the heart). The AV node 54 is the only electrical bridge that allows the impulses to travel from the atria 22, 42 to the ventricles 32, 46. The impulse travels through the walls of the ventricles 32, 46, causing them to contract. They squeeze and pump blood out of the heart. As mentioned above, the right ventricle 32 pumps blood to the lungs, and the left ventricle 46 pumps blood to the body. When the SA node 52 is directing the electrical activity of the heart, the rhythm is called “normal sinus rhythm.” A normal heart may beat about 60 to 100 times per minute at rest for a normal, regular rhythm.

Unfortunately, there are many people who suffer from or are at risk for irregular heart rhythm. One common type of irregular heart rhythm is supraventricular tachyarrhythmia (SVT). As an example, SVT may result from atrial fibrillation (AFib). During AFib, many different electrical impulses rapidly fire at once, rather than the SA node 52 regularly directing the electrical rhythm, causing a very fast, chaotic rhythm in the atria 22, 42. Because the electrical impulses are so fast and chaotic, the atria 22, 42 cannot contract and/or squeeze blood effectively into the ventricles 32, 46. During AFib, the many impulses beginning at the same time and spread through the atria, competing for a chance to travel through the AV node 54. The AV node 54 limits the number of impulses that travel to the ventricles 32, 46, but many impulses get through in a fast and disorganized manner. The ventricles 32, 46 contract irregularly, leading to a rapid and irregular heartbeat. During AFib, the rate of impulses in the atria can range from 300 to 600 beats per minute. This dangerously elevated heart rate can be referred to as atrial tachycardia (AT). Both atrial fibrillation (AFib) and atrial tachycardia (AT) are types of supraventricular tachyarrhythmia (SVT).

There is no one “cause” of atrial fibrillation, although it is associated with many conditions, including, but not limited to hypertension (high blood pressure), coronary artery disease, heart valve disease, post-heart-surgery recovery, chronic lung disease, heart failure, cardiomyopathy, congenital heart disease, pulmonary embolism, hyperthyroidism, pericarditis, and viral infection. In some people with AFib, no underlying heart disease is found. In these cases, AFib may be related to alcohol or excessive caffeine use, stress, certain drugs, electrolyte or metabolic imbalances, severe infections, or genetic factors. In some cases, no cause can be found. The risk of AFib increases with age, particularly after age 60.

Atrial fibrillation can lead to many problems. Since the atria are beating rapidly and irregularly during AFib, blood does not flow through them as quickly. This makes the blood more likely to clot. If a clot is pumped out of the heart, it can travel to the brain, resulting in a stroke. People with atrial fibrillation are 5 to 7 times more likely to have a stroke than the general population. Clots can also travel to other parts of the body (kidneys, heart, intestines), and cause other damage. Atrial fibrillation can decrease the heart's pumping ability. The irregularity can make the heart work less efficiently. In addition, atrial fibrillation that occurs over a long period of time can significantly weaken the heart and lead to heart failure.

Many patients suffering from or at risk for SVT opt to have an implantable cardioverter defibrillator (ICD), such as a pacemaker, installed in their body. Such implantable devices typically send small pacing electrical impulses to the heart muscle to maintain a suitable heart rate. Typically, if the pacing impulses are not effective, then the ICD shocks the heart with a larger defibrillation impulse in an attempt to force the heart back into a normal rhythm. Implantable cardioverter defibrillators (ICDs), such as pacemakers, have a pulse generator with one or more leads (wires) that send impulses from the pulse generator to the heart muscle, as well one or more leads to sense the heart's electrical activity.

While the pacing and/or defibrillation therapies provided by an ICD can be useful for patients with SVT (including AFib and/or atrial tachycardia), such therapies are often inappropriate for a separate type of irregular heart rhythm: ventricular tachyarrhythmia (VT). Ventricular tachyarrhythmia (VT) can include ventricular tachycardia (V-Tach), an abnormally fast heart rhythm that starts in the lower part of the heart (ventricles 32, 46). If left untreated, some forms of ventricular tachycardia (V-Tach) may get worse and lead to ventricular fibrillation (VF), which can be life-threatening. With ventricular fibrillation (VF), the heart may beat so fast and irregularly that the heart stops pumping blood. Ventricular fibrillation is a leading cause of sudden cardiac death. Both ventricular tachycardia (V-Tach) and ventricular fibrillation (VF) are types of ventricular tachyarrhythmia (VT).

Appropriate therapy for VT differs from the therapies used to treat SVT. Unfortunately, however, many implantable cardioverter defibrillator (ICDs) are not able to distinguish VT from SVT, leading to inappropriate therapy during either ventricular tachyarrhythmia (VT) or supraventricular tachyarrhythmia (SVT), depending on which diagnosis is the default diagnosis. For example, anti-tachycardia pacing (ATP) has been shown to successfully terminate VT in over 90% of cases making this a painless initial therapy in ICDs. As a result programing strategies usually employ a single sequence of ATP prior to delivery of a shock if the tachycardia is classified as stable despite the cycle length (CL) being recorded in a VF detection interval. If ATP fails to terminate the tachycardia, there is usually an escalation in therapies to defibrillation. Some manufacturers also resort to committed therapies after failure to terminate the episode based on the original diagnostic criteria. Thus, if there was an incorrect classification of a supra-ventricular tachycardia (SVT) at the outset, multiple inappropriate shocks may be delivered by the device. Although this misdiagnosis may occur in any type of ICD, the problem is more likely in single chamber ICDs were the absence of an atrial lead makes diagnosis of VT reliant on rudimentary discriminatory criteria (i.e. rapidity of onset and stability of the tachycardia) and in patients with AF. Some manufacturers also offer a morphology detection algorithm which has been shown to reduce inappropriate therapies, however, existing algorithms are prone to errors in sampling.

Therefore, there is a need for a reliable method and system for differentiating between supraventricular tachyarrhythmia (SVT) and ventricular tachyarrhythmia (VT) so that the incidence of inappropriate therapies therefor may be reduced or eliminated.

### SUMMARY

A method of differentiating between supraventricular tachyarrhythmia (SVT) and ventricular tachyarrhythmia (VT) is disclosed. A post pacing interval (PPI) is determined based on a biomarker dataset. The post pacing interval is statistically analyzed relative to a threshold to differentiate between SVT and VT.

A further method of differentiating between supraventricular tachyarrhythmia (SVT) and ventricular tachyarrhythmia (VT) is disclosed. A post pacing interval (PPI) is determined based on a biomarker dataset. A tachycardia cycle length (TCL) is determined based on the biomarker dataset. A difference of the PPI minus the TCL is statistically analyzed relative to a threshold to differentiate between SVT and VT.

Another method of differentiating between supraventricular tachyarrhythmia (SVT) and ventricular tachyarrhythmia (VT) is disclosed. The method comprises determining a post pacing interval (PPI) based on a biomarker dataset. In one embodiment the PPI is determined based on a biomarker dataset by determining a first return cycle length from a portion of the biomarker dataset which follows an episode of anti-tachycardia pacing (ATP). In one embodiment a mean tachycardia cycle length (TCL) is determined based on the biomarker dataset by averaging a plurality of cycle lengths after the first return cycle length from a portion of the biomarker dataset which precedes or follows the episode of anti-tachycardia pacing (ATP). In another embodiment a mean tachycardia cycle length (TCL) is determined based on the biomarker dataset by averaging a plurality of tachycardia cycle lengths which precedes or follows an episode of ATP. A difference of the PPI minus the mean TCL is statistically analyzed relative to a threshold to differentiate between SVT and VT. An appropriateness of a cardiac treatment protocol is determined based on the differentiation between SVT and VT.

In embodiments provided herein, the biomarker dataset comprises, but is not limited to, an R-peak to peak interval, a heart rate, a scatterplot (dot-plot), a heartbeat cycle time, or an electrogram (EGM). The electrogram data may be provided from an implanted ventricular sensor lead of an implantable cardioverter defibrillator (ICD). The scatterplot may be provided from an ICD.

A non-transitory computer readable medium is also disclosed. The non-transitory computer readable medium has stored thereon instructions for differentiating between supraventricular tachyarrhythmia (SVT) and ventricular tachyarrhythmia (VT), which, when executed by a processor, cause the processor to: a) determine a post pacing interval (PPI) based on a biomarker dataset; and b) statistically analyze the post pacing interval relative to a threshold to differentiate between SVT and VT.

A system is also disclosed for differentiating between supraventricular tachyarrhythmia (SVT) and ventricular tachyarrhythmia (VT). The system has a processor configured determine a post pacing interval (PPI) based on a biomarker dataset, and statistically analyze the PPI relative to a threshold to differentiate between SVT and VT. The system also has a data input coupled to the processor and configured to provide the processor with the biomarker dataset. The system further has a user interface coupled to either the processor or the data input.

It is at least one goal of the claimed invention to provide an improved method for differentiating between supraventricular tachyarrhythmia (SVT) and ventricular tachyarrhythmia (VT) so that the incidence of inappropriate therapies therefor may be reduced or eliminated.

### BRIEF DESCRIPTION OF THE DRAWINGS

For a better understanding of the invention, and to show more clearly how it may be carried into effect, embodiments will be described, by way of example, with reference to the accompanying drawings, wherein:

FIG. 1 schematically illustrates the operation of a human heart.

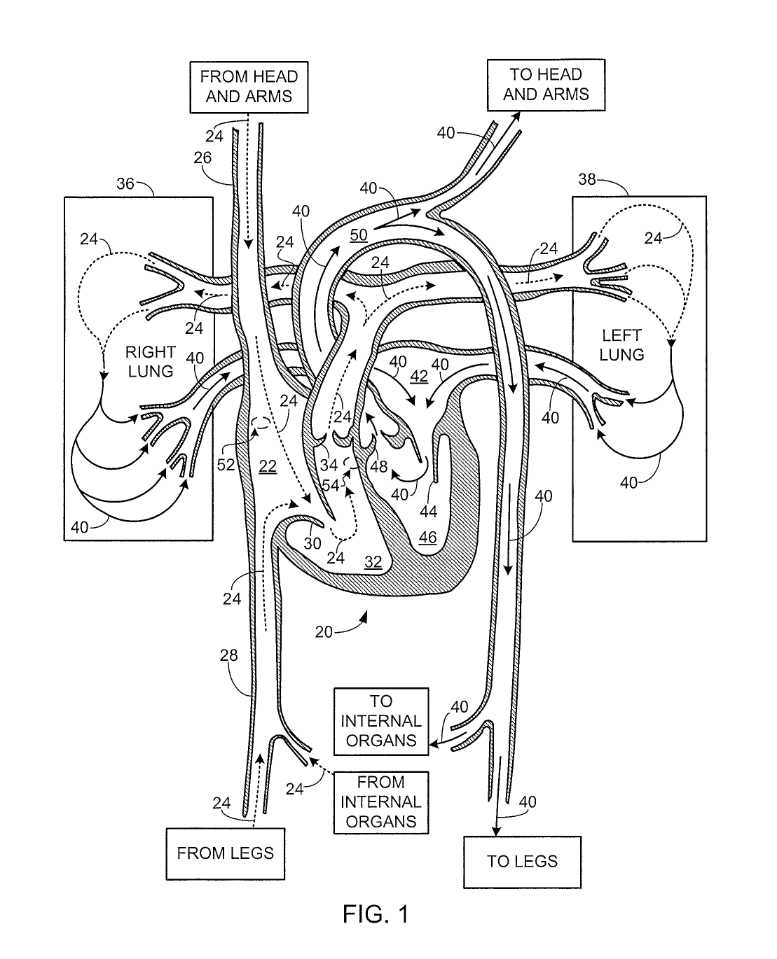

FIG. 2A schematically illustrates an embodiment of an electrogram (EGM) showing one heartbeat.

FIGS. 2B and 2C schematically illustrate an embodiment of an electrogram (EGM) showing multiple heart beats.

FIG. 3 illustrates one embodiment of a method for differentiating between supraventricular tachyarrhythmia (SVT) and ventricular tachyarrhythmia (VT).

FIG. 4 illustrates another embodiment of a method for differentiating between supraventricular tachyarrhythmia (SVT) and ventricular tachyarrhythmia (VT).

FIG. 5 schematically illustrates an embodiment of system 90 for differentiating between supraventricular tachyarrhythmia (SVT) and ventricular tachyarrhythmia (VT).

FIG. 6 schematically illustrates another embodiment of system 104 for differentiating between supraventricular tachyarrhythmia (SVT) and ventricular tachyarrhythmia (VT).

FIG. 7 schematically illustrates a further embodiment of system 110 for differentiating between supraventricular tachyarrhythmia (SVT) and ventricular tachyarrhythmia (VT).

FIG. 8 is an illustration of the ATP response with the PPI and TCL intervals in an episode of atrial tachycardia with a rapidly conducted ventricular rate resulting in inappropriately delivered ATP by a dual chamber device.

FIG. 9. illustrates distribution of the experimental data and the respective use of PPI as a discriminatory tool for SVT and VT.

FIG. 10 illustrates distribution of data and the respective means using PPI-TCL as a discriminatory tool for SVT and VT.

FIG. 11 illustrates the receiver operating characteristic (ROC) curves of PPI and PPI-TCL parameters shown together.

FIG. 12 is a diagrammatic representation of a pacing site at distance X from a macro-reentrant tachycardia utilizing a critical isthmus.

FIGS. 13A and 13B show simple linear plot of the absolute PPI and PPI-TCL values, respectively for AF/AT and VT showing minimal overlap.

FIGS. 14A and 14B illustrate a scatterplot and corresponding intracardiac EGM, respectively, depicting a burst of ATP terminating an episode of VT.

FIG. 15A illustrates an episode of rapidly conducted AF into the VT zone resulting in several rate driven inappropriate therapies illustrated in this scatter plot.

FIG. 15B illustrates the first ramp ATP before shocks results in a prolonged PPI of 660 ms.

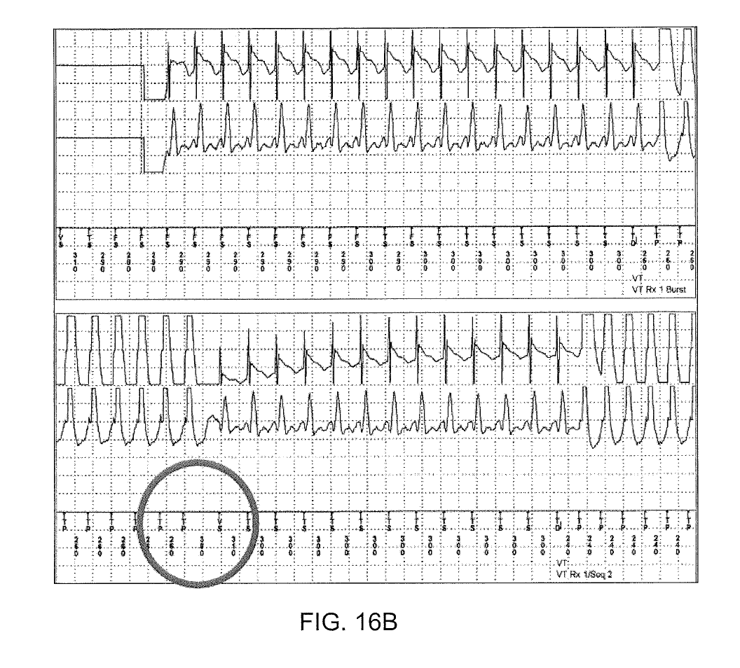

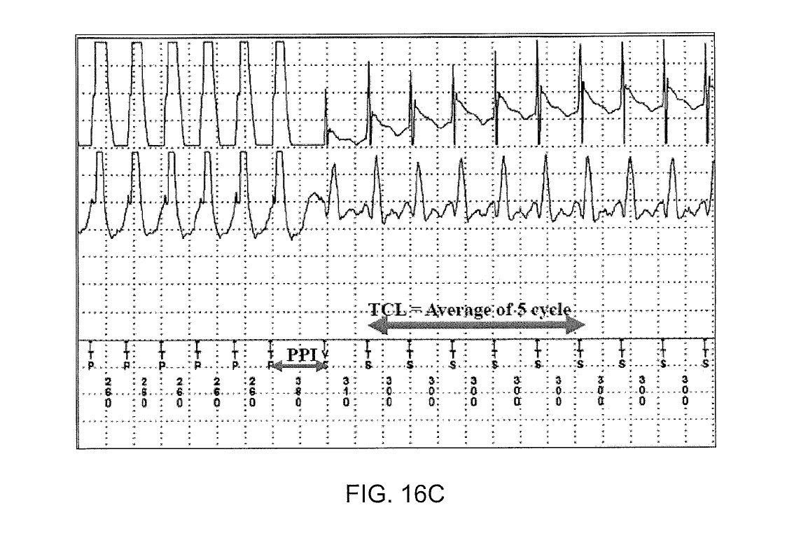

The PPI (arrows) has been amplified in FIG. 15C.

The scatterplot of FIG. 16A illustrates an arrhythmia detected in the single ventricular lead of an ICD.

The EGMs of FIG. 16B show that this return PPI is relatively short at 380 ms.

The PPI interval of FIG. 16C is amplified also with the PPI-TCL interval calculated as (380−300) ms=80 ms making VT the most likely diagnosis and therefore therapy is appropriate.

It will be appreciated that for purposes of clarity and where deemed appropriate, reference numerals have been repeated in the figures to indicate corresponding features, and that the various elements in the drawings have not necessarily been drawn to scale in order to better show the features.

### DETAILED DESCRIPTION

Many different types of biomarker data may be provided for the heart. Different non-limiting examples of heart biomarker data may include or be derived from measurement of the electrical activity of the heart. For example, an electrogram (EGM) such as a surface electrocardiogram or an intracardiac electrogram may be measured by an EGM capture device which can have one or more leads which are coupled to and/or implanted in a person's body in various locations. The electrical activity occurring within individual cells throughout the heart produces a cardiac electrical vector which can be measured by the one or more EGM capture device leads. Non-limiting examples of EGM capture devices include, but are not limited to, an implantable cardioverter defibrillator (ICD) having one or more leads implanted in the heart, or an external signal measurement device having one or more leads coupled to a patient's body. It should be understood that EGM capture devices of any number of leads may be used to gather a set of EGM signals for use as biomarkers.

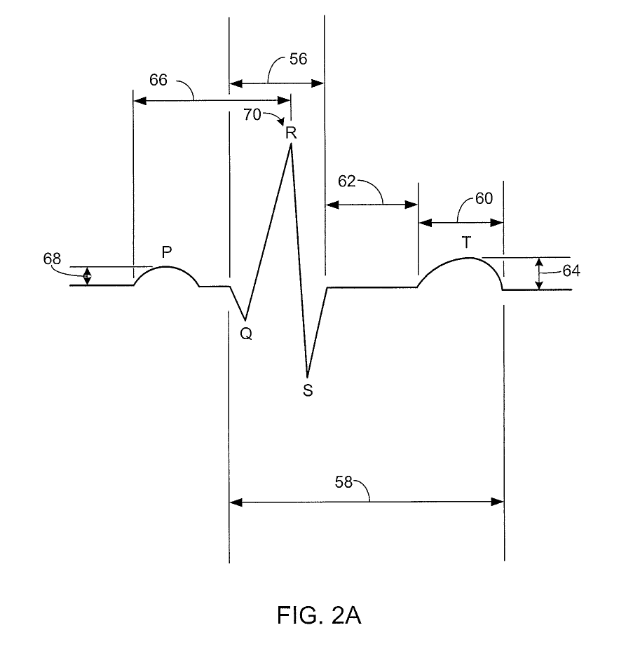

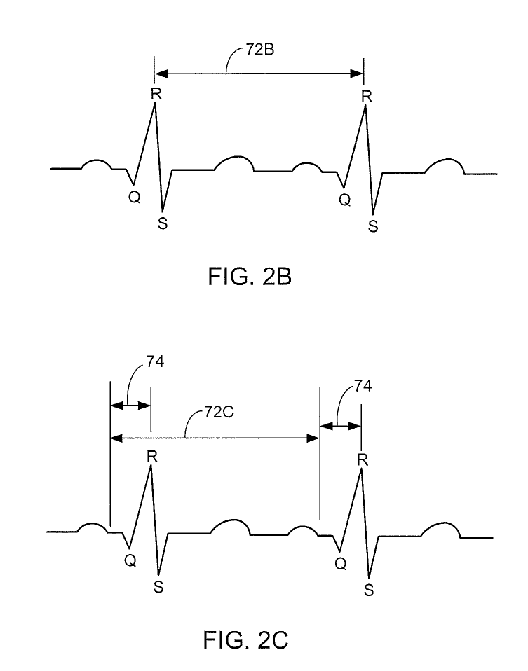

While an EGM signal itself could be considered a biomarker, other types of biomarker data may be derived from one or more EGM signals. For example, FIG. 2A schematically illustrates an embodiment of an EGM showing a single heart beat and some of the biomarkers which are commonly determined based on various portions of the EGM signal. The QRS complex 56 is associated with the depolarization of the heart ventricles. The QT interval 58 and the T-wave 60 are associated with repolarization of the heart ventricles. The ST segment 62 falls between the QRS complex 56 and the T-wave 60. Those skilled in the art will recognize that there are a multitude of available EGM-based biomarkers, and that this list is just provided as an example. Other non-limiting examples include the amplitude of the T wave 64, a PR interval 66, the amplitude of the P wave 68, and the peak 70 of the QRS complex (R-peak). When consecutive EGM beats are examined together, for example those schematically illustrated in FIGS. 2B and 2C, a cycle time 72B, 72C between adjacent heart beats can be determined. In the example of FIG. 2B, the cycle time 72B is measured from the R-peak of a first heart beat to the R-peak of the following heartbeat. R-peak is readily identifiable on the EGM, and therefore it may be useful in determination of the cycle time between adjacent heart beats. In other embodiments, the cycle time may be determined based on a position in the heart beats that is relative to the R-peak. For example, a cycle time 72C is measured in the embodiment schematically illustrated in FIG. 2C from a time 74 preceding the R-peak in consecutive heart beats. In still other embodiments, the cycle time may be determined based on positions in the heart beat which are not based on R-peak.

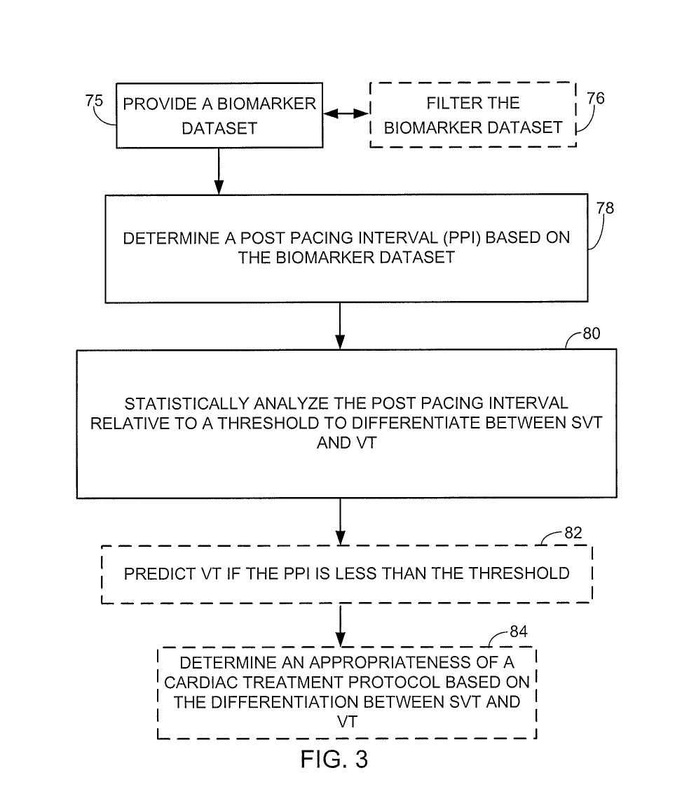

FIG. 3 illustrates one embodiment of a method for differentiating between supraventricular tachyarrhythmia (SVT) and ventricular tachyarrhythmia (VT). A biomarker dataset is provided 75. Non-limiting examples of suitable biomarkers have been discussed above. For simplicity, a biomarker dataset derived from EGM signals is discussed in more detail for this embodiment. However, it should be understood that other types of biomarker datasets may be used, and the data may be raw data or processed data. Raw data may be, for example, EGM data, and processed data may be, for example, a scatterplot (dotplot). The biomarker dataset may also comprise, but is not limited to, an R-peak to peak interval, a heart rate, or a heartbeat cycle time. The biomarker dataset may comprise the results of previous EGM signal analysis. If using EGM signals, the EGM signals may be provided from a variety of implantable and non-implantable EGM capture devices as discussed above, for example, a ventricular ICD lead. The EGM signals may be provided in “real-time” from a subject coupled to an EGM capture device, or the EGM signals may be provided from a database (which should be understood to include memory devices) storing previously obtained EGM signals. In some embodiments, the biomarker dataset may optionally be filtered 76. One suitable method of filtering EGM signals is to apply digital low-pass finite impulse response (FIR) filtering to remove baseline wandering. Another suitable method of filtering EGM signals to remove baseline wander is to subtract a baseline estimation arrived-at using spline interpolation. In other embodiments, the optional filtering 76 may include the discarding of one or more leading beats. In other embodiments, one or more trailing beats may be discarded.

In step 78, a post pacing interval (PPI) is determined based on the biomarker dataset. The PPI is the first return cycle length after anti-tachycardia pacing (ATP) ends. Therefore, PPI may be considered a cycle length measured from the last heart beat which resulted from electrical pacing provided by an ICD to the first ensuing heart beat which the heart generates on its own after pacing is ended. This PPI cycle length can be measured based on a variety of corresponding parts of the two heart beats, for example, from R-peak to R-peak as illustrated in FIG. 2B or from some other corresponding position as illustrated in FIG. 2C.

In step 80, the PPI is statistically analyzed relative to a threshold to differentiate between SVT and VT. As will be discussed in more detail in the experimental data section later in this disclosure, it has been discovered that ventricular tachyarrhythmia (VT) may be predicted 82 if the PPI is less than a threshold. If the PPI is not less than the threshold, and if exclusion criteria, such as those outlined in the later experiments are not triggered, then SVT may be predicted. Based on the ability to differentiate between SVT and VT, an appropriateness of a cardiac treatment protocol may be determined 84. For example, heavy shocking of the heart or defibrillation may be avoided in cases where pacing was not effective and it was determined through the disclosed method that SVT is present (for example, atrial fibrillation or atrial tachycardia).

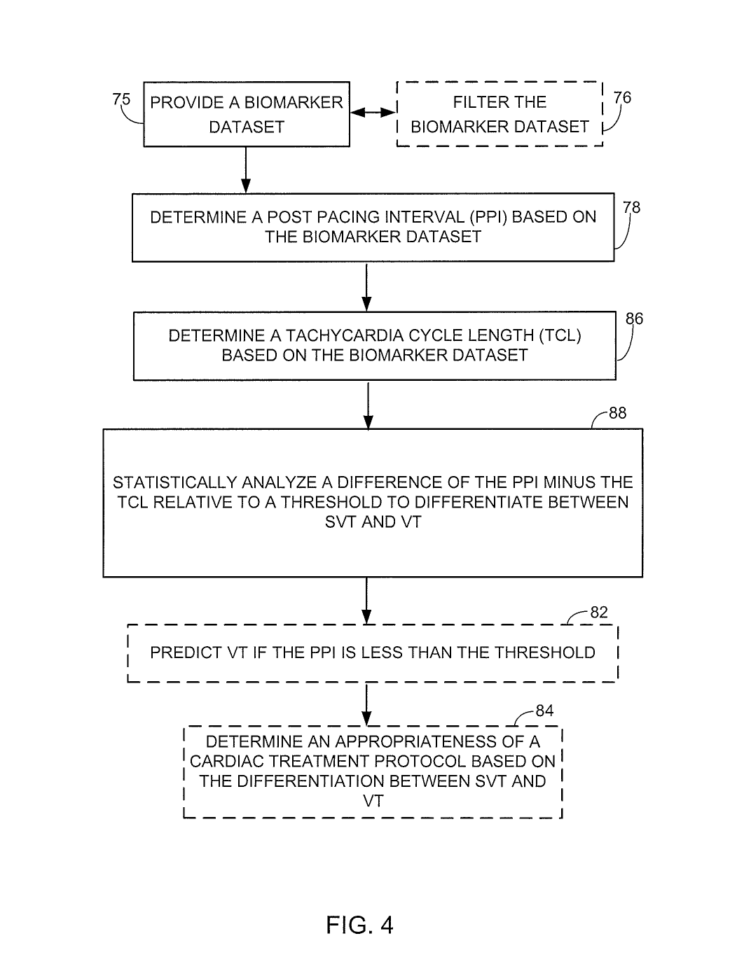

FIG. 4 illustrates another embodiment of a method for differentiating between supraventricular tachyarrhythmia (SVT) and ventricular tachyarrhythmia (VT). A biomarker dataset is provided 75. Non-limiting examples of suitable biomarkers have been discussed above. For simplicity, a biomarker dataset derived from EGM signals is discussed in more detail for this embodiment. The EGM signals may be provided in “real-time” from a subject coupled to an EGM capture device, or the EGM signals may be provided from a database (which should be understood to include memory devices) storing previously obtained EGM signals. As discussed previously, in some embodiments, the biomarker dataset may optionally be filtered 76.

In step 78, a post pacing interval (PPI) is determined based on the biomarker dataset. The PPI is the first return cycle length after anti-tachycardia pacing (ATP) ends. Therefore, PPI may be considered a cycle length measured from the last heart beat which resulted from electrical pacing provided by an ICD to the first ensuing heart beat which the heart generates on its own after pacing is ended. This PPI cycle length can be measured based on a variety of corresponding parts of the two heart beats as discussed above.

In step 86, a tachycardia cycle length (TCL) is determined based on the biomarker dataset. Depending on the embodiment, this TCL may be determined from one or more heartbeats of the biomarker dataset after the first return cycle following the end of the anti-tachycardia pacing (ATP). If the TCL determination is made from more than one heartbeat, then an average or other desired statistical combination of the multiple beats may be used to determine the mean TCL, depending on the embodiment.

In step 88, a difference of the PPI minus the TCL (e.g., the mean TCL) is statistically analyzed relative to a threshold to differentiate between SVT and VT. As will be discussed in more detail in the experimental data section later in this application, it has been discovered that ventricular tachyarrhythmia (VT) may be predicted 82 if the difference of the PPI minus and the mean TCL is less than a threshold. If the PPI minus mean TCL difference is not less than the threshold, and if exclusion criteria, such as those outlined in the later experiments are not triggered, then SVT may be predicted. Based on the ability to differentiate between SVT and VT, an appropriateness of a cardiac treatment protocol may be determined 84. For example, heavy shocking of the heart or defibrillation may be avoided in cases where pacing was not effective and it was determined through the disclosed method that SVT is present (for example, atrial fibrillation or atrial tachycardia). Alternative methods may include normalizing the PPI and/or subjecting the PPI data to a mathematical operation, or determining a ratio of PPI to mean TCL.

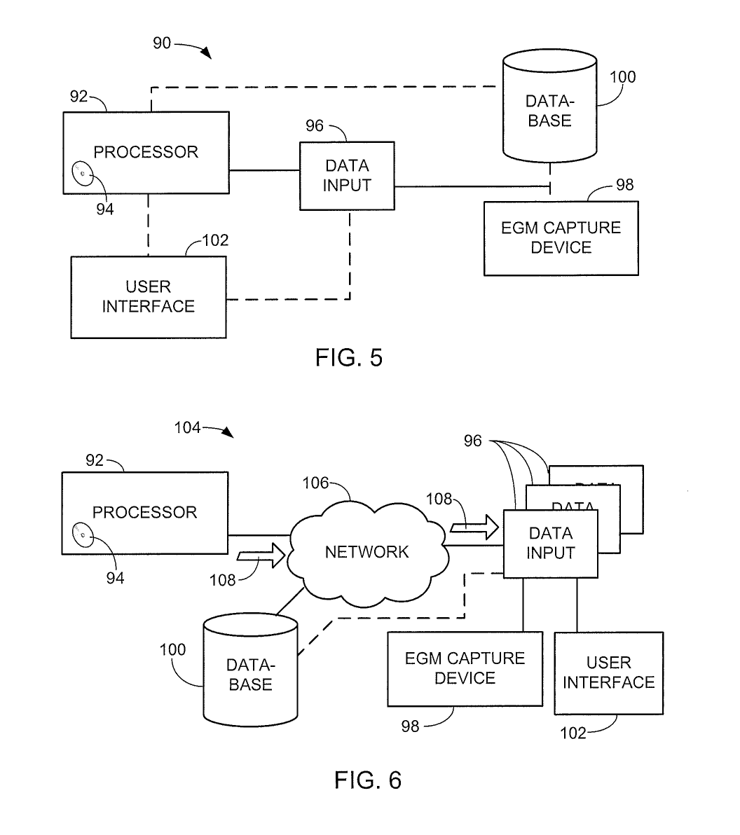

FIG. 5 schematically illustrates an embodiment of system 90 for differentiating between supraventricular tachyarrhythmia (SVT) and ventricular tachyarrhythmia (VT). The system 90 has a processor 92 which is configured to determine a post pacing interval (PPI) based on a biomarker dataset, and statistically analyze the PPI relative to a threshold to differentiate between SVT and VT. Embodiments of suitable processes and method steps to make this determination have already been discussed above. The processor 92 may be a computer executing machine readable instructions which are stored on a non-transitory computer readable medium 94, such as, but not limited to a CD, a magnetic tape, an optical drive, a DVD, a hard drive, a flash drive, a memory card, a memory chip, or any other computer readable medium. The processor 92 may alternatively or additionally include a laptop, a microprocessor, an application-specific integrated circuit (ASIC), digital components, analog components, or any combination and/or plurality thereof. The processor 92 may be a stand-alone unit, or it may be a distributed set of devices.

A data input 96 is coupled to the processor 92 and configured to provide the processor with EGM biomarker data. An EGM capture device 98 may optionally be coupled to the data input 96 to enable the live capture of EGM biomarker data. Examples of EGM capture devices include, but are not limited to, a ventricular lead of an ICD device, an atrial lead, a twelve-lead EGM device, an eight-lead EGM device, a two lead EGM device, a Holter device, a bipolar EGM device, and a uni-polar EGM device. Similarly, a database 100 may optionally be coupled to the data input 96 to provide previously captured EGM signal biomarker data to the processor 92. Database 100 can be as simple as a memory device holding raw data or formatted files, or database 100 can be a complex relational database. Depending on the embodiment, none, one, or multiple databases 100 and/or EGM capture devices 98 may be coupled to the data input 96. The EGM capture device 98 may be coupled to the data input 96 by a wired connection, an optical connection, or by a wireless connection. Suitable examples of wireless connections may include, but are not limited to, RF connections using an 802.11x protocol or the Bluetooth® protocol. The EGM capture device 98 may be configured to transmit data to the data input 96 only during times which do not interfere with data measurement times of the EGM capture device 98. If interference between wireless transmission and the measurements being taken is not an issue, then transmission can occur at any desired time. Furthermore, in embodiments having a database 100, the processor 92 may be coupled to the database 100 for storing results or accessing data by bypassing the data input 96.

The system 90 also has a user interface 102 which may be coupled to either the processor 92 and/or the data input 96. The user interface 102 can be configured to display the EGM signal biomarker data, a determination of PPI, TCL, and/or TCL-PPI, and a determination of the appropriateness of a cardiac treatment protocol. The user interface 102 may also be configured to allow a user to select EGM signal biomarker data from a database 100 coupled to the data input 96, or to start and stop collecting data from an EGM capture device 98 which is coupled to the data input 96.

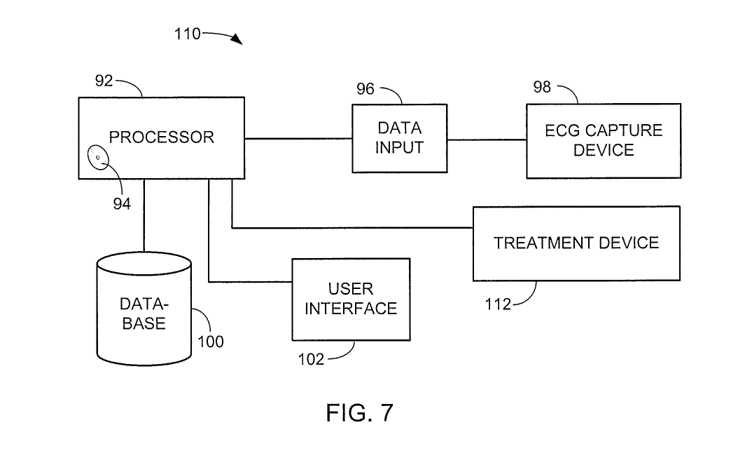

FIG. 6 schematically illustrates another embodiment of system 104 for differentiating between supraventricular tachyarrhythmia (SVT) and ventricular tachyarrhythmia (VT). In this embodiment, the processor 92 is set-up to be a remote processor which is coupled to the data input 96 over a network 106. The network 106 may be a wired or wireless local area network (LAN or WLAN) or the network 106 may be a wired or wireless wide area network (WAN, WWAN) using any number of communications protocols to pass data back and forth. Having a system 104 where the processor 92 is located remotely allows multiple client side data inputs 96 to share the resources of the processor 92. EGM signal biomarkers may be obtained by the data input 96 from a database 100 and/or an EGM capture device 98 under the control of a user interface 102 coupled to the data input 96. The EGM signal biomarker data may then be transferred over the network 106 to the processor 92 which can then differentiate between supraventricular tachyarrhythmia (SVT) and ventricular tachyarrhythmia (VT) and transmit data signals 108 having the predicted clinical outcome to the client side. Such data transmissions may take place over a variety of transmission media, such as wired cable, optical cable, and air. In this embodiment, the remote processor 92 can be used to help keep the cost of the client-side hardware down, and can facilitate any upgrades to the processor or the instructions being carried out by the processor, since there is a central upgrade point.

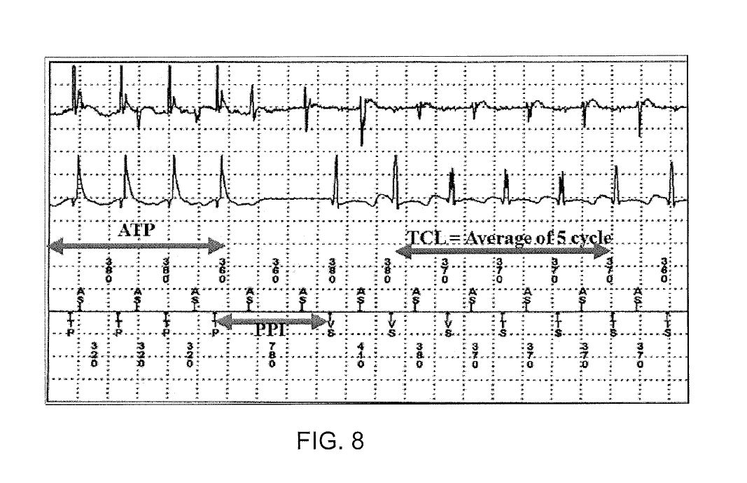

FIG. 7 schematically illustrates a further embodiment of system 110 for differentiating between supraventricular tachyarrhythmia (SVT) and ventricular tachyarrhythmia (VT). In this embodiment, a data input 96, a user interface 102, and a database 100 are coupled to the processor 92. An EGM capture device 98 is coupled to the data input 96. The system 110 also has a treatment device 112 which is coupled to the processor 92. The treatment device 112 may be configured to administer a pharmacological agent, electrical pacing, and/or a defibrillation shock to a patient when enabled by the processor 92. The system 110 of FIG. 7, and its equivalents, may be useful in automating pharmacological and/or electrical treatments for the heart based on the VT versus SVT differentiation made possible by the methods disclosed herein and their equivalents.

Methods for differentiating between supraventricular tachyarrhythmia and ventricular arrhythmia, such as those discussed above, have been used in validations with encouraging results to identify whether or not an associated treatment is appropriate.

### Experimental Results:

This study was a retrospective analysis of all patients implanted with ICDs for combined primary and secondary indications at a single center. The cohort consisted of 250 patients (46 female). These were of mixed ischaemic and non-ischaemic aetiologies with a mean age of 73±7 years. All patients received either dual chamber (DR) or biventricular (BiV) ICDs. Patients were excluded if they received single chamber devices or if the atrial ports of the devices were plugged. This was done so that only episodes with a corresponding atrial electrogram (EGM) would be analyzed for proof of concept. Events were adjudicated by two observers. The maximum follow up period was 23 months.

### Data Collection

The clinical records of all patients implanted with DR and BiV ICDs, were examined for the period December 2006-October 2008. All patient related device therapies that were flagged in a database were then re-examined. These were then classified into appropriate and inappropriate therapies (ITS). All non-physiological events (“noise related events”) and oversensing phenomena were excluded from the analysis.

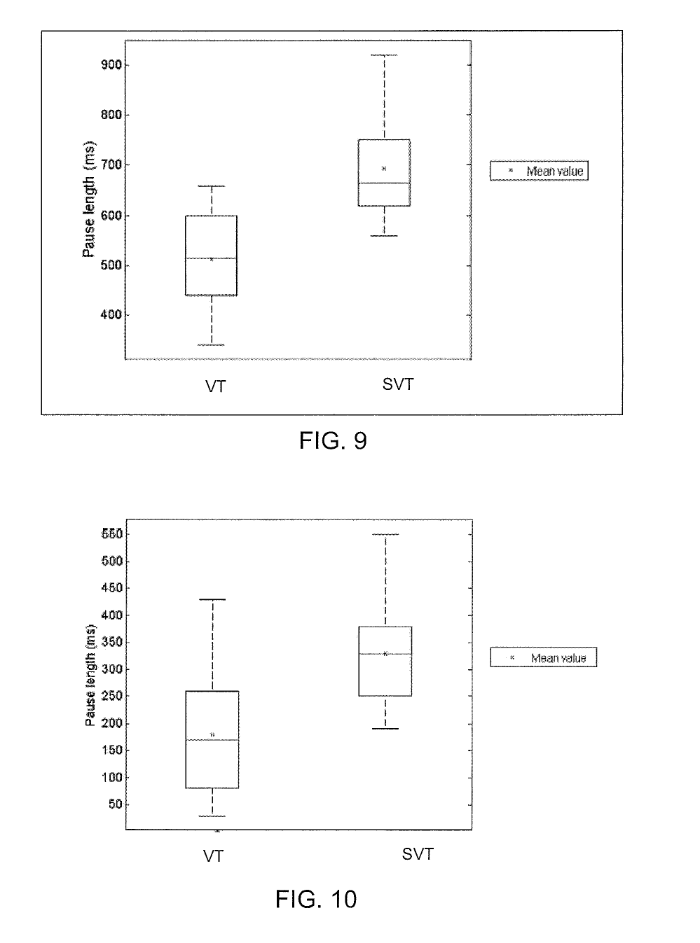

The post pacing interval (PPI) was defined as the first return cycle length after ATP (burst/ramp). The PPI-TCL was determined where TCL was the average ventricular cycle length calculated by the device in this embodiment. The mean cycle length of the tachycardia for analysis was determined in this embodiment as the average of 5 successive cycle lengths, excluding the first interval after the PPI (to allow for minor CL variation after ATP). FIG. 8 is an illustration of the ATP response with the PPI and TCL intervals in an episode of atrial tachycardia with a rapidly conducted ventricular rate resulting in inappropriately delivered ATP by a dual chamber device. The tachycardia continues after ATP is seen to dissociate the ventricle from the atrium during pacing yielding a “pseudo atrial-atrial-ventricular (AAV)” post ATP response.

In this embodiment, the mean CL post ATP was compared to the preceding mean CL of the tachycardia pre ATP to ensure that there was no significant variation in tachycardia (i.e. CL variation >50 ms) (refer to exclusion criteria listed below).

### Exclusion Criteria

In this embodiment, exclusion criteria were applied to the remaining data sets to ensure that the delivered ATP did not significantly perturb the ongoing tachycardia to account for the episode as a single ongoing event. The exclusion criteria for this experiment embodiment included:

1. The post ATP and pre ATP TCL varied >50 ms if the preceding tachycardia was stable, e.g., in VT, AT or pseudo regularization of rapidly conducted AF.

2. ATP terminated the episode.

3. A ventricular paced event occurred at the lower programed rate immediately after ATP.

4. ATP accelerated the preceding tachycardia CL by >50 ms.

All Events were Evaluated in a Standardized Manner:

a. The scatter plot (dot-plot) and local and farfield EGMs were viewed collectively to aid diagnosis. b. Events were evaluated in chronological order (episode) and then sequentially (sequence of ATP) per individual patient. c. Each episode was categorized as appropriate or inappropriate depending if criteria for VT or AF/AT were observed (Table 1).

TABLE 1
Criteria for distinguishing VT from SVT using
device scatter plots and intracardiac EGMs

| Ventricular Tachyarrhythmia | Supraventricular Tachyarrhythmia |
|---|---|
| (VT) | (SVT) |
| Onset of tachycardia in ventricle | Onset of tachycardia in atrium |
| Ventricular Rate > Atrial Rate | Atrial Rate > Ventricular Rate |
| Atrial and Ventricular dissociation | Ventricular Rate dependent on |
| | Atrial Rate |
| Stable Ventricular-Ventricular | Number of Atrial Events > Number |
| relationship | of Ventricular Events |
| Ventricular to Atrial timing with | Unstable/variable Ventricular- |
| retrograde conduction | Ventricular relationship |
| | Variable Ventricular to Atrial timing |

### Statistical Analysis

Patient demographics are represented as mean±standard deviation. Data was analyzed and presented using Minitab and SPSS. In this embodiment, a t-test was used to assess significance and a p<0.05 was regarded as significant. Receiver operator curves (ROC) were used to determine absolute cutoff values that would differentiate AF/AT from VT.

### Results

There were 165 DR and 85 BiV ICDs implanted in the cohort. All Medtronic® ICDs, for example, Entrust®, Virtuoso® and later models were included because of familiarity of the observers with the manufacturer's method of data representation allowing optimal observer diagnostic capability.

A total of 39 episodes were identified in 20 patients over a mean follow up period of 23 months. The incidence of delivered therapies was 8% in this population. There were 76 sequences of ATP (burst/ramp) delivered in total within these episodes. Twenty eight sequences (37%) were categorized as inappropriately delivered for either AF/AT. This was observed despite all but one patient having advanced discriminators turned on. All elements of Medtronic's PR logic algorithm (Medtronic Inc., Minn., USA) were selected except for “other 1:1 SVTs” which is nominally turned off in these devices.

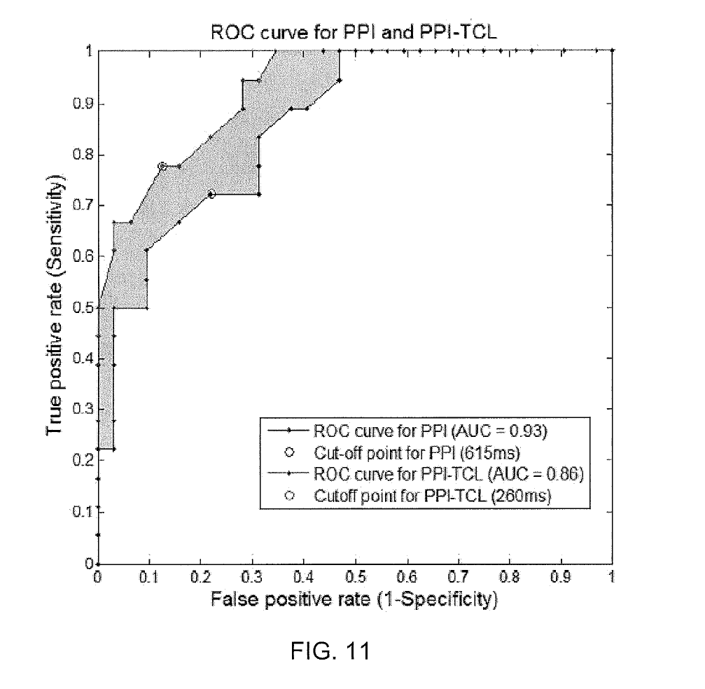

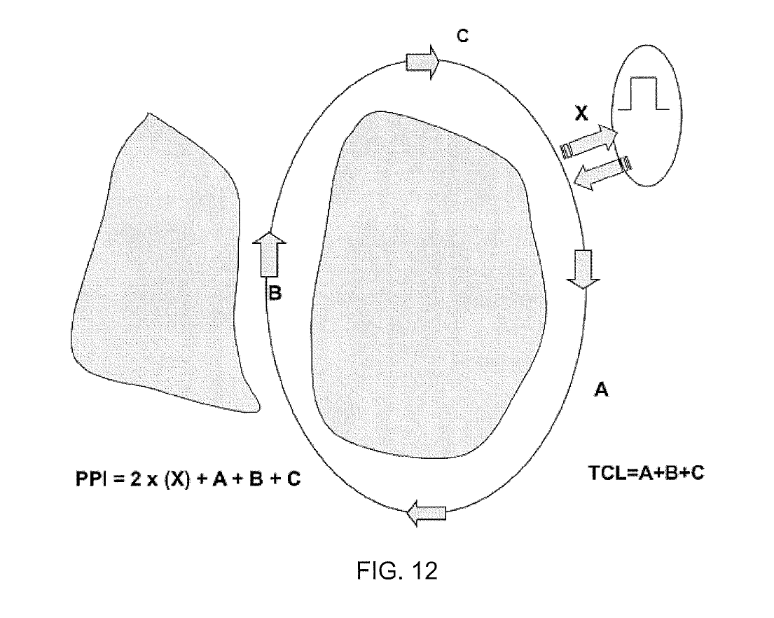

After applying the exclusion criteria listed above, 51 sequences of ATP (n=18 AT/AF, n=33 VT) were available for analysis. The mean PPI was 693±96 ms vs 582±83 ms, p<0.01 and mean PPI-TCL was 330±97 ms vs 179±102 ms, p<0.01 for AT/AF and VT respectively. FIG. 9. illustrates distribution of the experimental data and the respective use of PPI as a discriminatory tool for SVT and VT. FIG. 10 illustrates distribution of data and the respective means using PPI-TCL as a discriminatory tool for SVT and VT.

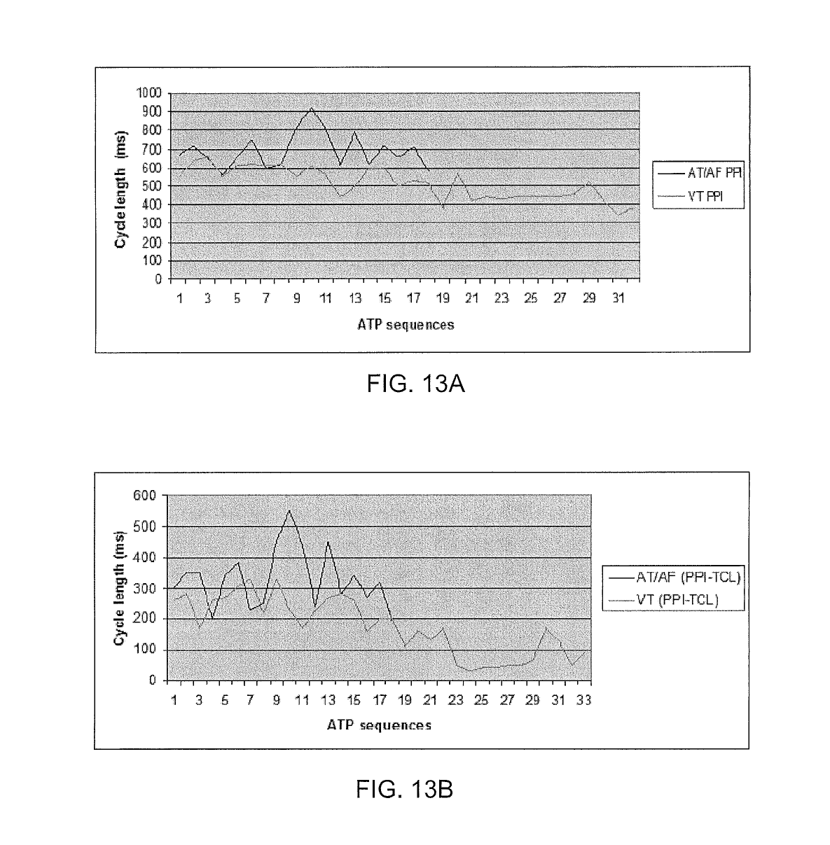

A ROC curve was applied to both the PPI and PPI-TCL diagnostic criteria to determine an absolute cutoff to that would define VT from conducted AF/AT (SVT). Cutoffs of about 615 ms, area under the curve (AUC) 0.93 (95% confidence interval (CI): 0.84-1.00), p<0.01 for the PPI and about 260 ms, AUC 0.86 (95% CI: 0.74-0.98), p<0.01 for PPI-TCL (See FIG. 11) were identified. FIG. 11 illustrates the ROC curves of PPI and PPI-TCL parameters shown together. The PPI parameter appears to be more robust with a greater area under the curve (shaded portion).

When applying the above two criteria, a PPI<615 ms predicted VT rather than AF/AT with a sensitivity of 77.8% (95% CI: 58.6%-97.0%) and a specificity of 87.5% (95% CI: 76.0%-99.0%). The positive predictive value (PPV) for AT/AF detection was 77.8% (95% CI 58.6%-97%) and the negative predictive value (NPV), i.e., not AT/AF but VT, was 87.5% (95% CI 76.0%-99.0%).

PPI-TCL with values <260 ms was more likely to be VT than AF/AT with a sensitivity of 72.2% (95% CI: 51.5%-92.9%) and a specificity of 78.1% (95% CI: 63.8%-92.4%). The PPV was 72.2% (95% CI 51.5%-92.9%) for AT/AF and the NPV was 78.1% (95% CI 63.8%-92.4%), i.e., not AF/AT but VT detected. Thus the predictive value of both parameters had a greater likelihood of diagnosing VT than AF/AT which is acceptable for a default setting within an ICD where the primary aim is geared towards detecting and treating VT (or VF).

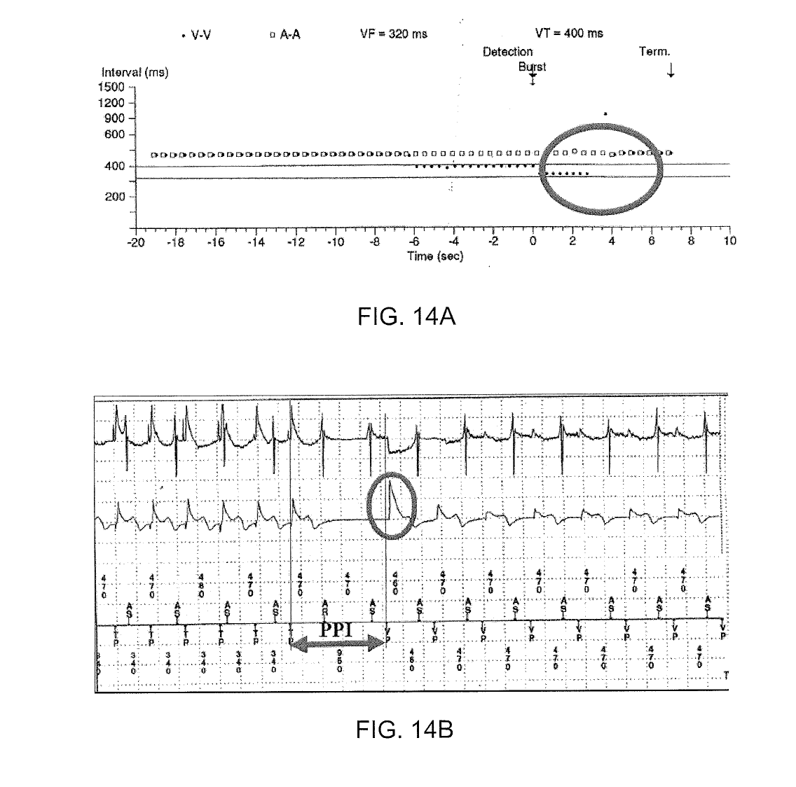

The above mentioned study analyses the PPI after a failed episode of ATP until resumption of sensed ventricular intervals by the ICD. Entrainment of a macro re-entrant tachycardia is indicative of the proximity of the pacing electrode to the circuit. FIG. 12 is a diagrammatic representation of a pacing site at distance X from a macro-reentrant tachycardia utilizing a critical isthmus. The tachycardia cycle length is a sum of limbs A+B+C. The post pacing interval (PPI) would therefore represent 2×X+A+B+C as the pacing stimulus would need to penetrate the re-entrant circuit and return to the site of pacing. A PPI<30 ms generally denotes that the pacing stimulus is directly within the macro-reentrant circuit (FIG. 12). This was not the case for cases with VT (mean PPI 582±83 ms) or for AF/AT (mean PPI 693±96 ms) as the site of the re-entrant substrate was very likely to be situated in the left ventricle particularly for the ischemic cardiomyopathies.

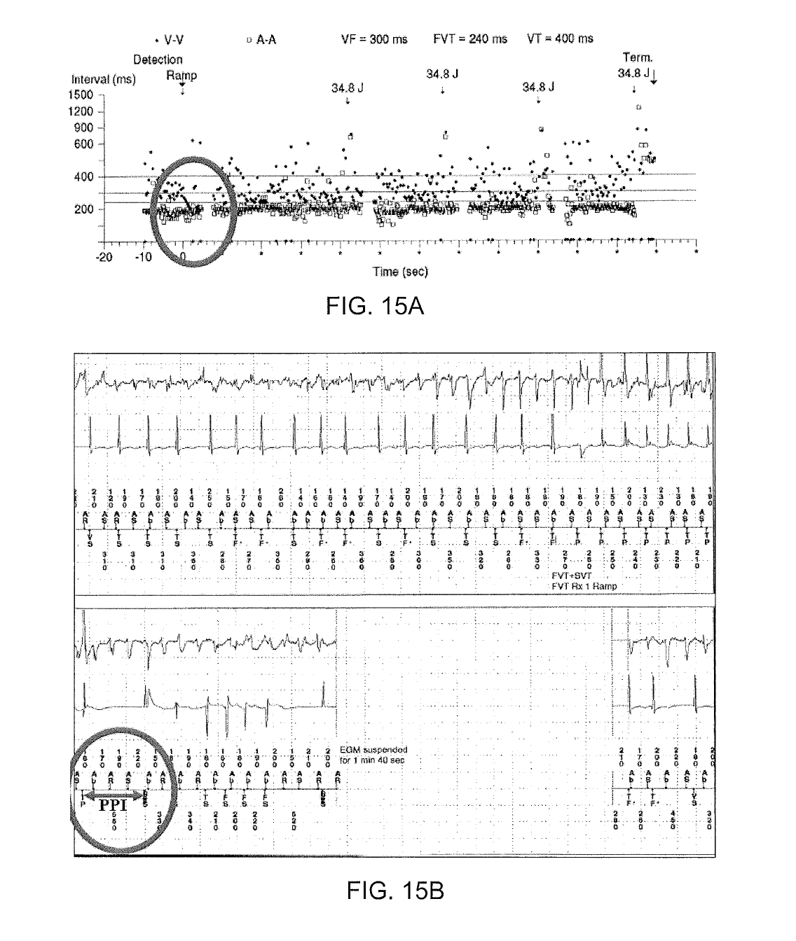

In these embodiments, all ICD implants involved lead placement at the right ventricular apex. Anti-tachycardia pacing was only from the RV electrode in DR ICDs and from both the RV and LV leads in BiV devices. The fact that both ventricles were paced in patients with BiV defibrillators would not affect the principle of using the PPI and PPI-TCL values for VT and AF/AT discrimination. The ROC curves provide an absolute value where a PPI<615 ms and a PPI-TCL<260 ms was more likely to be VT (See FIG. 11). However, when reviewing the distribution of actual recorded PPI and PPI-TCL values, there were areas of overlap. For example, FIGS. 13A and 13B show simple linear plot of the absolute PPI and PPI-TCL values, respectively for AF/AT and VT showing minimal overlap. This may be accounted for possibly because of the re-entrant substrate being located in the basal-lateral segment of the LV. The distance from the pacing source in the RV would therefore approximate interval recordings in AT/AF after retrograde invasion of the His-Purkinje system.

A high attrition rate (33%) was recorded for this data in this study. This was because of the strict exclusion criteria that were set in order to maintain the integrity of the measured parameters. These have been defined above.

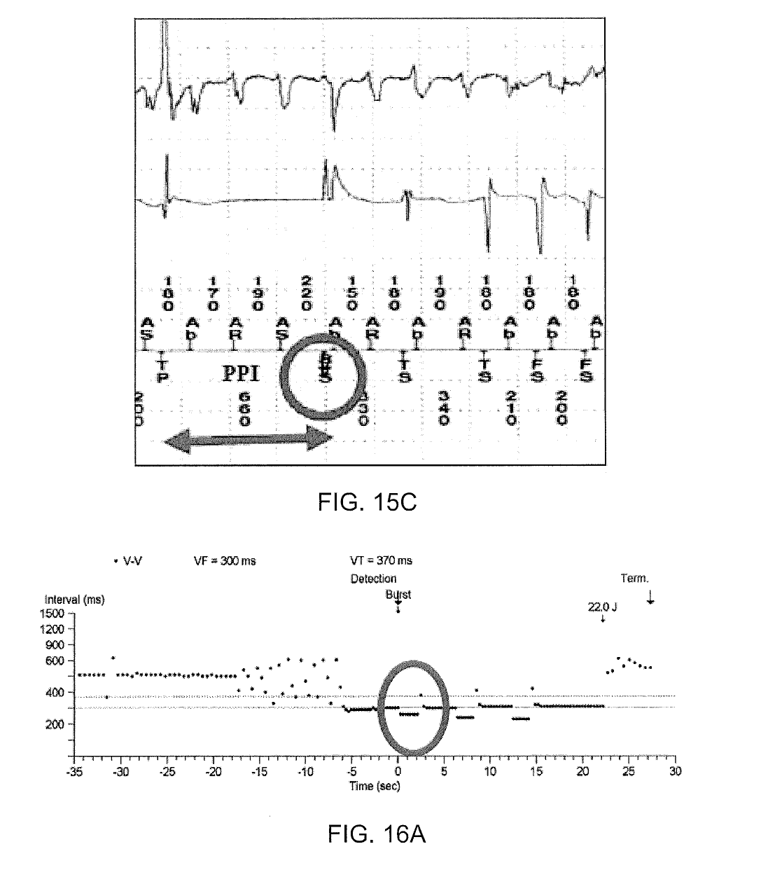

FIGS. 14A and 14B illustrate a scatterplot and corresponding intracardiac EGM, respectively, depicting a burst of ATP terminating an episode of VT. Note the dissociation of ventricular events (dots) from atrial events (boxes). The EGM of the ringed area in the scatterplot is depicted here. The first beat thereafter (ringed) shows onset of pacing (VP) thus yielding a falsely long PPI. This episode was therefore excluded from the analysis. In FIGS. 14A and 14B, an episode of VT is terminated with a resultant pause encroaching within the lower pacing rate of the device. Pacing then continues with the sinus rate being tracked with sequential ventricular pacing in a dual chamber (DDD) mode. Termination of VT and resumption of pacing, even for one beat, were regarded as an exclusion from analysis.

Once a tachycardia is classified as VT or VF and therapy is initiated and if criteria for episode termination are not fulfilled, subsequent therapies may be committed. This phenomenon in some manufacturers may perpetuate and even escalate therapies for an originally misclassified rhythm disorder, which is illustrative of the need for the disclosed and claimed invention.

FIG. 15A illustrates an episode of rapidly conducted AF into the VT zone resulting in several rate driven inappropriate therapies illustrated in this scatter plot. FIG. 15B illustrates the first ramp ATP before shocks results in a prolonged PPI of 660 ms. (This is ringed in A with corresponding EGMs in FIG. 15B).

The PPI (arrows) has been amplified in FIG. 15C. The first beat of conducted AF after the PPI has a biventricular pacing (BV) output superimposed. This is an “evoked sense response” from the biventricular ICD which attempts to resynchronize with pacing on the first beat. This is a normal function in this biventricular ICD. The long PPI is indicative of a conducted supraventricular arrhythmia namely, AF in this case. Rapidly conducted AF (FIGS. 15A-15C) results in detection within the VF zone. Various ATP modalities including burst and ramp programming were unsuccessful in terminating the tachycardia hence the device proceeds to shock which also failed to terminate the AF initially until the fourth shock resolves the AF to a slower AT or sinus tachycardia with conducted ventricular rates below the tachycardia detection interval (TDI). Each ATP whether burst/ramp is characterized by long PPI and PPI-TCL intervals in keeping with a tachycardia with a supraventricular origin. This suggests that these criteria can be applied after initial therapy (in this case painless ATP) to abort progression to shock. The application of the PPI and PPI-TCL parameters are therefore proposed as “downstream” criteria in the decision making tree in devices and therefore would not affect the initial points of entry into, manufacturer specific, existing software. This concept can be used by those skilled in the art when evaluating EGMs in patients presenting with shocks in order to decide if they were in fact appropriate or not.

The scatterplot of FIG. 16A illustrates an arrhythmia detected in the single ventricular lead of this device. The chamber of origin of the tachycardia is uncertain as there is no atrial EGM. Burst ATP entrains ventricle without termination of the arrhythmia (ringed). The EGMs of FIG. 16B show that this return PPI is relatively short at 380 ms. The PPI interval of FIG. 16C is amplified also with the PPI-TCL interval calculated as (380−300) ms=80 ms making VT the most likely diagnosis and therefore therapy is appropriate. The failed ATP sequence is escalated to a subsequent, more aggressive sequence of ATP and eventually will lead to a shock if these sequences fail. Although these experiments were conducted with multi-lead devices, other embodiments could utilize single chamber devices where the absence of an atrial lead makes such ICDs more prone to result in inappropriate treatments and also where it is difficult to interpret the intracardiac EGMs with only ventricular event recordings available.

The PPI and PPI-TCL difference are electrophysiological concepts that indicate proximity of a pacing site to the source of a tachycardia. This concept was applied to differentiate V-tach/VF (VT) from AT/AF (SVT) showing significant differences in the mean values for both these tachycardia sources when identified in DR and BiV ICDs. Although this concept was proven in devices with preexisting atrial leads, it has application as a discriminator in single chamber ICDs with or without morphologic discriminators. It also allows device specialists an additional modality to help interpret difficult ICD derived EGMs when deciding if the delivered therapy was appropriate or not. It has the potential to be incorporated into future implantable devices as a downstream application to re-evaluate the result of the ATP to avoid progression to a shock or committed therapies in the case of inappropriate therapies for AF/AT (SVT).

### EQUIVALENTS

Embodiments discussed have been described by way of example in this specification. It will be apparent to those skilled in the art that the forgoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and the scope of the claimed invention. Additionally, the recited order of processing elements or sequences, or the use of numbers, letters, or other designations therefore, is not intended to limit the claims to any order, except as may be specified in the claims. Accordingly, the invention is limited only by the following claims and equivalents thereto.

1. A method of differentiating between supraventricular tachyarrhythmia (SVT) and ventricular tachyarrhythmia (VT), comprising:
a) determining a post pacing interval (PPI) based on a biomarker dataset; and
b) statistically analyzing the post pacing interval relative to a threshold to differentiate between SVT and VT.

2. The method of claim 1, wherein the biomarker dataset comprises cardiac electrogram (EGM) data.

3. The method of claim 1, further comprising providing a biomarker dataset.

4. The method of claim 3, wherein providing the biomarker dataset comprises sampling EGM data provided from one or more implanted sensor leads from an implantable cardioverter defibrillator (ICD).

5. The method of claim 4, wherein at least one of the one or more implanted sensor leads comprises a ventricular ICD lead.

6. The method of claim 1, wherein the determining the post pacing interval (PPI) comprises determining a first return cycle length from a portion of the biomarker dataset which follows an episode of anti-tachycardia pacing (ATP).

7. The method of claim 1, wherein the biomarker dataset comprises at least one member selected from the group consisting of:
an R-peak to peak interval;
a heart rate;
a scatterplot;
a heartbeat cycle time; and
an electrogram (EGM).

8. The method of claim 1, wherein statistically analyzing the post pacing interval relative to the threshold to differentiate between SVT and VT comprises predicting VT if the PPI is less than the threshold.

9. (canceled)

10. The method of claim 1, further comprising determining a tachycardia cycle length (TCL) based on the biomarker dataset; and
wherein statistically analyzing the post pacing interval relative to the threshold to differentiate between SVT and VT comprises predicting VT if a difference of PPI minus TCL is less than the threshold.

11. (canceled)

12. The method of claim 10, wherein determining the TCL comprises determining a mean TCL by averaging a plurality of tachycardia cycle lengths which precede or follow an episode of anti-tachycardia pacing (ATP).

13. (canceled)

14. The method of claim 1, further comprising determining an appropriateness of a cardiac treatment protocol based on the differentiation between SVT and VT.

15. The method of claim 14, wherein:
the cardiac treatment protocol comprises a cardiac defibrillation; and
determining the appropriateness of the cardiac treatment based on the differentiation between SVT and VT comprises determining that the cardiac defibrillation is inappropriate when SVT is predicted.

16. The method of claim 1, further comprising instigating a cardiac treatment protocol based on the differentiation between SVT and VT.

17-36. (canceled)

37. A non-transitory computer readable medium having stored thereon instructions for differentiating between supraventricular tachyarrhythmia (SVT) and ventricular tachyarrhythmia (VT), which, when executed by a processor, cause the processor to:
a) determine a post pacing interval (PPI) based on a biomarker dataset; and
b) statistically analyze the post pacing interval relative to a threshold to differentiate between SVT and VT.

38. The non-transitory computer readable medium of claim 37, wherein the biomarker dataset comprises cardiac electrogram (EGM) data.

39. The non-transitory computer readable medium of claim 37, wherein the instructions further cause the processor to provide a biomarker dataset.

40. The non-transitory computer readable medium of claim 39, wherein the instructions to provide the biomarker dataset comprise instructions to sample EGM data provided from one or more implanted sensor leads from an implantable cardioverter defibrillator (ICD).

41. The non-transitory computer readable medium of claim 40, wherein at least one of the one or more implanted sensor leads comprises a ventricular ICD lead.

42. The non-transitory computer readable medium of claim 37, wherein the instructions to determine the post pacing interval (PPI) comprise instructions to determine a first return cycle length from a portion of the biomarker dataset which follows an episode of anti-tachycardia pacing (ATP).

43. The non-transitory computer readable medium of claim 37, wherein the biomarker dataset comprises at least one member selected from the group consisting of:
an R-peak to peak interval;
a heart rate;
a scatterplot;
a heartbeat cycle time; and
an electrogram (EGM).

44. The non-transitory computer readable medium of claim 37, wherein the instructions to statistically analyze the post pacing interval relative to the threshold to differentiate between SVT and VT comprise instructions to predict VT if the PPI is less than the threshold.

45. (canceled)

46. The non-transitory computer readable medium of claim 37, further comprising instructions to determine a tachycardia cycle length (TCL) based on the biomarker dataset; and
wherein the instructions to statistically analyze the post pacing interval relative to the threshold to differentiate between SVT and VT comprise instructions to predict VT if a difference of PPI minus TCL is less than the threshold.

47. (canceled)

48. The non-transitory computer readable medium of claim 46, wherein the instructions to determine the TCL comprise instructions to determine a mean TCL by averaging a plurality of cycle lengths which precede or follow an episode of anti-tachycardia pacing (ATP).

49. (canceled)

50. The non-transitory computer readable medium of claim 37, further comprising instructions to determine an appropriateness of a cardiac treatment protocol based on the differentiation between SVT and VT.

51. The non-transitory computer readable medium of claim 50, wherein:
the cardiac treatment protocol comprises a cardiac defibrillation; and
the instructions to determine the appropriateness of the cardiac treatment based on the differentiation between SVT and VT comprise instructions to determine that the cardiac defibrillation is inappropriate when SVT is predicted.

52. The non-transitory computer readable medium of claim 37, further comprising instructions to instigate a cardiac treatment protocol based on the differentiation between SVT and VT.

53-80. (canceled)
